(19) United States
(12) Patent Application Publication
(10) Pub. No.: US 2023/0404082 A1
(43) Pub. Date: Dec. 21, 2023

(54) AGRICULTURAL COMPOSITION COMPOSED BY CONSORTIUM BETWEEN Azospirillum sp. and Pseudomonas sp., PRODUCTION PROCESS AND INCREASED STABILITY THEREOF, AND USE AS PROMOTOR OF PLANT GROWTH FOR AGRICULTURAL APPLICATION

(71) Applicant: TOTAL BIOTECNOLOGIA INDUSTRIA E COMERCIO S.A., Curitiba (BR)

(72) Inventors: Josiane FUKAMI, Vinhedo (BR); Douglas Fabiano GOMES, Vinhedo (BR); Juliana Marcolino GOMES, Vinhedo (BR); Jonas Hipolito DE ASSIS FILHO, Vinhedo (BR)

(21) Appl. No.: 18/251,824

(22) PCT Filed: Sep. 15, 2021
(86) PCT No.: PCT/BR21/50394

§ 371 Date: May 4, 2023

(51) Int. Cl. A01N 63/27; A01N 63/20; A01P 21/00

(52) U.S. Cl. CPC A01N 63/27; A01N 63/20; A01P 21/00

(57) ABSTRACT

The present invention refers to an agricultural composition that comprises a mixture of Azospirillum brasilense and Pseudomonas fluorescens in a single commercial inoculant product that promotes increased stability and cellular viability due to the synergism between these microorganisms, and which surprisingly increases the shelf-life achieved by a differentiated industrial process. The industrial process of the mixture of Azospirillum and Pseudomonas as product inoculant plant growth promoting is composed by steps of: (a) fermentation of the microorganisms to obtain an agricultural composition with plant growth promotion action through biological mechanisms to fix nitrogen, biosynthesis of auxins, siderophores and solubilization of phosphorus; (b) stabilization of the product biotechnological inoculant composed by the mixture of Azospirillum and Pseudomonas in a technical solution that enables the mixture of the parts, presented in a single package. The synergism demonstrated is mainly connected to (i) the high production of EPS by Azospirillum which is consumed by Pseudomonas as energy source and (ii) to the production of organic acids by Pseudomonas which enables the maintenance of cellular viability of the Azospirillum.

### FIELD OF THE INVENTION

The present invention comprises increased stability and, consequently, shelf-life, of an agricultural composition comprised by a consortium of plant growth promoting microorganisms, Azospirillum sp. and Pseudomonas sp. The invention presented contemplates the industrial process and potential agricultural application thereof.

### BACKGROUND OF THE INVENTION

The growth predictions of the world population require an increase in agricultural production to meet the demand for quality foods. Over recent decades, agricultural productivity has increased due to the use of genetically-modified plant varieties and application of chemical supplies, be they to protect pests and diseases or nutrition. As a consequence of the excessive consumption of inappropriate chemicals and agricultural practices, the deterioration of the chemical, physical and biological properties of the farmable soils limit the yield of agriculturally important crops. Therefore, agriculture now faces the challenge of increasing the quantity and quality of what it produces, repairing the damage caused by inappropriate management. Therefore, efficient and sustainable agricultural practices need to be developed and implemented with a view to controlling phytopathogens and increasing yields without the need for opening up new farmable areas, which almost always occurs through deforestation (Yadav et al., 2017).

Another factor that negatively impacts the yields of the main agricultural crops are climactic changes, which occur in an accelerated rhythm as a result of uncontrolled emissions of greenhouse gases, added to environmental degradation. Waves of heat and drought repeat cycle upon cycle leading to significant losses in productivity in the main agriculturally important crops (Altenbach 2012). To a certain extent, the use of chemical supplies in agribusiness contributes to greenhouse gas emissions, including nitrous oxide (N2O), generated by the process of nitrification/denitrification of ammonia (Kool et al., 2011), nitrogen fertilizer widely used in agriculture.

New strategies designed to increase agricultural productivity and reduce impacts generated by the conventional practices are in evidence, particularly those focused on regenerating the microbial biodiversity of the soils, this being crucial for restoring and boosting sustainability of agricultural production systems. Along these lines, biotechnology applied to agriculture has received major efforts for developing innovative solutions that contribute to the sustained management of economically important crops. Among these solutions, the use of microorganisms that deliver benefits to the plants or to the agricultural system appears as an important innovation tool. This approach uses bacteria and fungi that associate to the plant roots and cause beneficial effects in the vegetable development through direct or indirect action mechanisms, these microorganisms being called plant growth promoting (Hill et al., 2000; Yadav et al., 2015; Verma et al., 2016; Kumar et al., 2017).

The term “plant growth promoting bacteria (PGPB)” was first used by Klopper and Schroth (1978) to describe soil bacteria that colonize plant roots and increase their growth, and may survive and grow at low temperatures and pH, below 20° C. and pH 6.0. These bacteria have been extensively studied over recent years, which has generated important results on the performance mechanisms and culminate in improved vegetable development. Among the various genera of prominent microorganisms characterized as PGPB are Agrobacterium, Allorhizobium, Arthrobacter, Azospirillum, Azotobacter, Bacillus, Bradyrhizobium, Burkholderia, Caulobacter, Chromobacterium, Erwinia, Exiguobacterium, Flavobacterium, Mesorhizobium, Micrococcous, Providencia, Pseudomonas, Rhizobium and Serratia (Yadav et al., 2017; Suman et al., 2015; Suman et al., 2016).

The characterization of the microorganisms as promoting plant growth involves identifying one or more action mechanisms, the main being the solubilization of phosphorus (Pikovskaya, 1948), potassium (Hu and Guo, 2006) and zinc (Fasim et al., 2002), the production of phytohormones as auxins (Bric et al., 1991) and gibberellins (Brown, 1968), the fixing of nitrogen (Boddey et al., 1995) and the production of the ACC-deaminase enzyme (Jacobson et al., 1994). Further attributes of the bacteria that benefit plants are the biosynthesis of ammonia (Cappucino and Sherman, 1992), HCN (Bakker and Schippers, 1997), siderophores (Schwyn and Neilands, 1987) and the antagonist action to phytopathogens. When a species is characterized by presenting one or various paths that lead to the promotion of plant growth, it is studied in association with agricultural-interest cultures, measuring the parameters related to increased yield, chiefly productivity. Another important factor in the study of new microorganisms is the inoculation strategy, including inoculation via seed, plantation furrow or leaf spraying. Lastly, after validating the growth promoting action, the microorganisms become biotechnological targets for the development of products destined for agricultural application, seeking the optimal use of the natural resources, greater security for operators and reduction or replacement of chemical compounds, including fertilizers and pesticides.

In Brazil, various products present as active ingredient strains of Azospirillum brasilense, a plant growth promoting rhizobacteria characterized by biologically fixing nitrogen and biosynthesis of auxins (Molina et al., 2018). Azospirillum brasilense (Ab-V5 and Ab-V6), presents a cell in vibrio format, with an approximate diameter of 1 μm, Gram-negative, with average to high mobility, thanks to the presence of polar scourge. The colonies present 1 to 3 mm in diameter, circular format, are flat, with whole edges, smooth surface, slightly mucous, aqueous consistency, opaque, with red chromogenesis in CR medium (Cassán et. al., 2010). Various studies prove the efficient growth promoting action performed by this bacterium associated to the rhizosphere of different cultures, including corn (Zea mays), soybean (Glycine max) and bean (Phaseolus vulgaris). As main benefit, the application of Azospirillum enables chemical fertilization to be reduced for the supply of nitrogen by at least 25%, thus decreasing the costs of the agricultural activity and the environmental impacts related to the use of chemical fertilizers.

Another species broadly reported as promoting plant growth is Pseudomonas fluorescens. The P. fluorescens (CCTB03) is a rod-shaped bacterium, straight or slightly curved, with moderate mobility and variable size, Gram-negative. The colonies present typical fluorescence when cultivated in KingB medium (King et al., 1954) and lit under ultraviolet or regular light, wrinkled aspect, opaque, irregular edges, and variable size. This microorganism acts by different fronts benefitting the development and yield of important crops for agriculture. Examples of this species have already been reported for producing phytohormones (Duca et al., 2018), siderophores (Duffy and Defago, 1999) and ACC-deaminase (Shaharoona et al., 2006). These multiple functions assist in the development of the plant roots, which thereafter access hydric and nutritional resources with greater efficiency, in the acquisition of iron, an important enzymatic cofactor, and in overcoming these biotic and abiotic stresses due to the degradation of the ethylene precursors, a hormone related to a vegetable senescence. Another important biological role of P. fluorescens consists of the mobilization and solubilization of phosphorus through the biosynthesis of enzymes (phosphatases) and organic acids. This important characteristic brings the plants relevant amounts of this nutrient, one of the limiting factors to high productivity.

The proven efficiency of promoting plant growth in and of itself is no guarantee of success of the biotechnological application of a microorganism, and the preparation of products for agricultural application is a complex process involving various steps. Among them, stabilization is of the utmost importance, since the microorganisms need to be brought to the agricultural environment with high cellular viability so they can perform their functions, including those related to the promotion of plant growth.

In Brazil, legislation requires that a biological product destined for agriculture has a minimum guarantee of six month's storage, maintaining a minimum concentration of cells which, when applied, can perform their functions in benefit of the target cultures. Therefore, upholding cellular viability of the microorganisms in commercial products is a major challenge, which needs to be overcome so that the success of the technology is achieved to the fullest extent. The difficulty is even greater when a technology seeks the combination of different species in a single product, as is the case sought in this present draft; preparing a commercial inoculant product that promotes plant growth, composed of, but not limited to, Azospirillum brasilense and Pseudomonas fluorescens.

When biological products composed of viable cells from different species of bacteria are proposed, the complexity in meeting the nutritional and physiological requirements of each one of the active ingredients must be considered. The formulation of the culture medium is essential for directing the metabolism of the microorganisms for the biosynthesis of compounds that provide the compatibility and synergy between the species. The exopolysaccharides (EPS) of microbial origin, composed mainly of polysaccharides, proteins, nucleic acids and lipids (Flemming and Wingender, 2010), are broadly recognized for acting to uphold the viability of different species of bacteria (KopyciAska et al., 2018). Another compound, used as source of carbon by various microorganisms, the organic acids, for example malic acid, succinic acid, propionic acid and lactic acid, are important for upholding the viable cells for prolonged periods. In this context, the present draft describes the formulation, the industrial process and the results demonstrating that the polysaccharides produced in a directed way by A. brasilense and the organic acids induced during the multiplication of P. fluorescens act to extend the shelf-life and cellular viability of these two species combined in a product for agricultural application.

Surprisingly, the technical solution presented raises the cellular viability, and consequently, storage for prolonged periods, assuring the viability of biotechnological application of Azospirillum and Pseudomonas in a single solution with performance growth promoting superior to the products formulated with the isolated microorganisms.

### SUMMARY OF THE INVENTION

The present invention teaches that, surprisingly, it is possible to develop a biotechnological solution (on an industrial scale) containing two genera of microorganisms, Azospirillum and Pseudomonas in a single product, with high stability. Surprisingly, the mixture of the microorganisms and the induction of the synergism between different genera increases the shelf-life thereof through an industrial process that promotes the biosynthesis of EPS and organic acids, by Azospirillum and Pseudomonas, respectively.

The present invention further provides an agricultural composition produced by the method of the present invention, as well as the use thereof in agriculture.

Advantageously, the present invention enables the use of the agricultural composition with application in quantities of 150 mL/ha via seed, 300 to 1000 mL/ha via plantation furrow and 500 to 1000 mL/ha via leaf spraying.

Advantageously, the present invention enables an agricultural composition to be obtained with increased stability of the product, when compared to products composed by the same microorganisms formulated in isolation.

Also, surprisingly, the present invention provides additional parameters for the method of producing an agricultural composition constituted by two different genera of microorganisms on an industrial scale, demonstrating the parameters needed for the growth, such as parameters of pressure, temperature, oxygenation (volume of air and agitation) and the components of the culture medium, enabling a biotechnological product to be obtained.

As will be understood by a person skilled in the art, different species of Azospirillum and Pseudomonas may be used, as well as different parameters of fermentation and composition of the cultivation medium can be combined for the present invention.

In a first embodiment, the present invention provides a process of producing an agricultural composition, called inoculant, comprising the steps of:
(a) fermentation of the microorganisms in bioreactors for obtaining the inoculant with plant growth promotion action mainly, but not restricted to, through mechanisms for biologically fixing nitrogen, biosynthesis of auxins, siderophores and solubilization of phosphorus;
(b) formulation of a commercial biotechnological inoculant product composed of two genera of microorganisms in a technical solution that enables the mixture of the parts with high stability, presented in a single package.

Surprisingly, the preferred embodiment of the present invention is the mixture of two different genera of microorganisms produced on an industrial scale which, when packaged in consortium constituting a single product, presents increased stability.

Unexpectedly, the product with the two microorganisms together in a single package promotes the extension of the stability for at least 12 months, which corresponds to an increase of at least two-fold when compared to the product packaged with the individualized microorganisms (stable for periods under six months).

The synergism achieved with the interaction between the two microorganisms in a single product derives from the formation of floccules or aggregates, both associated to the production of exopolysaccharides (EPS) in Azospirillum, and of the biosynthesis of organic acids by Pseudomonas. The EPS are mainly composed of polysaccharides, proteins, nucleic acids and lipids. Advantageously, bacteria of the genus Pseudomonas were benefitted by presenting the capacity to consume these sources of carbon, nitrogen and fatty acids for maintaining the cellular viability inside the product.

In compliment to the synergistic actions between this consortium of microorganisms, the biosynthesis of organic acids by Pseudomonas was quantified and, provably, consumed by Azospirillum, increasing the stability of this microorganism in the agricultural composition maintaining the viability of the cells for prolonged periods.

Advantageously, the consumption of organic acids as source of carbon by Azospirillum generates an additional effect that promotes the stability of the agricultural composition, contributing to the adjustment of the pH along the storage period of the product, an essential characteristic for upholding the cellular viability of the components of the microbial consortium.

### BRIEF DESCRIPTION OF THE FIGURES

For improved understanding of the invention, reference should now be made to the embodiments of the invention illustrated in greater detail in the accompanying drawings and described by means of the embodiments of the invention.

FIG. 1 illustrates the synergism of Azospirillum and Pseudomonas in relation to the concentration of exopolysaccharides (EPS) and concentration of organic acids in different storage times for products composed by the microorganisms bottled in consortium.

The FIG. 2 illustrates the standard calibration curve with different concentrations of propionic acid (0.5-4 mM) as an indirect method of quantifying the production of organic acids produced by microorganisms.

The FIG. 3 illustrates the effect of the microorganisms bottled individually relative to the concentration of exopolysaccharides (EPS) and concentration of organic acids at different storage times.

### DETAILED DESCRIPTION OF THE INVENTION

In a preferred embodiment, according to the present invention, in step (a), the fermentation of the Azospirillum brasilense and Pseudomonas fluorescens per batch occurs for approximately 18 to 120 hours.

In a preferred embodiment, the method of the present invention comprises the sequential expansion (grading) of the culture of A. brasilense and P. fluorescens for inoculating the fermentation culture. Preferably, the sequential expansion is initiated in volumes of 100 mL, which serves as inoculum for about two flasks with volumes of 10 L. This, in turn, is inoculated in tanks with about 180 L of culture, which, ultimately, are transferred to reactors containing about 1600 L.

In a preferred embodiment, the A. brasilense and the P. fluorescens are expanded in flasks of about 100 mL of the NFb medium (Dobereiner, 1995) and 4.1 medium (Table 1), respectively, by incubation in orbital agitator to about 80 rpm to about 200 rpm. The incubation time is preferably about 18 hours to about 96 hours. Preferably, the microorganisms are then inoculated in inox flasks containing about 10 L of culture medium 4.4 (Table 2) and 4.1 (Table 1) for the A. brasilense and P. fluorescens, respectively. The incubation time is preferably about 18 to about 96 hours with air flow of about 0.25 Nm³/h to about 1.5 Nm³/h (=0.45-2.5 vvm).

TABLE 1
CULTURE MEDIUM USED FOR GROWTH OF P. FLUORESCENS
UP TO THE SCALE OF 200 L.

| | Reagents | | |
|---|---|---|---|
| 01 | K₂HPO₄ | 0.4-4 | g |
| 02 | KH₂PO₄ | 0.4-4 | g |
| 03 | (NH₄)₃PO₄ | 0.1-2 | g |
| 04 | MgSO₄•7H₂O | 0.1-2 | g |
| 05 | NaCl | 0.001-1 | g |
| 06 | KNO₃ | 0.5-5 | g |
| 07 | Yeast Extract | 0.5-5 | g |
| 08 | Solution MnSO4 (10%) | 10-100 | μL |
| 10 | Solution FeCl₃ (10%) | 10-100 | μL |
| 11 | Glycerol | 1-20 | mL |

| | | |
|---|---|---|
| 12 | Water | q.s. 1 L |

q.s.: quantum sufficit

TABLE 2
CULTURE MEDIUM USED FOR GROWTH OF
A. BRASILENSE UP TO SCALES OF 180 L.

| | Reagents | | |
|---|---|---|---|
| 01 | K₂HPO₄ | 1-10 | g |
| 02 | KH₂PO₄ | 0.5-5 | g |
| 03 | CaCl₂•2H₂O | 0.01-1 | g |
| 04 | MgSO₄•7H₂O | 0.2-2 | g |
| 05 | NaCl | 0.01-1 | g |
| 06 | NH₄Cl | 1-10 | g |
| 07 | Yeast Extract | 1-10 | g |
| 09 | KOH | 30-100 | g |
| 10 | Solution of micronutrients*¹ | 1-10 | mL |
| 11 | Fe-EDTA | 0.01-1 | g |
| 12 | Malic acid | 1-10 | g |

| | | |
|---|---|---|
| 13 | Water | q.s. |

q.s.: quantum sufficit
*¹see table 4.

In a preferred embodiment, the culture temperature for multiplication according to the present invention is about 22° C. to about 38° C.

In a preferred embodiment, A. brasilense and P. fluorescens are inoculated separately in the process of grading up to 180 L and mixed in the fermenters of 2000 L as described for the present invention. Accordingly, in a preferred embodiment, said inocula of 100 mL are inoculated in two flasks made of inox of about 10 L and then transferred in tanks containing about 180 L of specific culture medium for each microorganism, incubated for about 24 to about 168 hours. The air flow is, preferably, about 0.25 to about 1.0 Nm³/h (=0.45-2.5 vvm).

In a preferred embodiment, the tank of the A. brasilense is then inoculated in fermenters of 2000 L and the culture is carried out at a temperature of about 22° C. to about 38° C. The air flow is preferably about 1.0 Nm³/h to about 2.5 Nm³/h (=0.0085-0.021 vvm). The fermentation time is preferably about 18 to about 120 hours. The pressure is preferably about 0.5 to about 1.2 kgf/cm 2. Agitation is preferably from about 30 hz to about 45 hz. Preferably, the A. brasilense is conducted for a period of 18 to 120 h.

In a preferred embodiment, at the end of the fermentation process of the A. brasilense, a tank containing 200 L of P. fluorescens is then inoculated and mixed to the fermenter of 2000 L.

In a preferred embodiment, according to the present invention, in step (b), the formulation used for the agricultural composition that enables the mixture of two microorganisms in fermenters of 2000 L is described as per Table 3.

TABLE 3
CULTURE MEDIUM USED FOR GROWTH OF
MICRORGANISMS IN FERMENTER 2000 L.

| | Reagents | | |
|---|---|---|---|
| 01 | K₂HPO₄ | 1-10 | g |
| 02 | KH₂PO₄ | 0.5-5 | g |
| 03 | CaCl₂•2H₂O | 0.01-1 | g |
| 04 | MgSO₄•7H₂O | 0.2-2 | g |
| 05 | NaCl | 0.01-1 | g |
| 06 | NH₄Cl | 1-10 | g |
| 07 | Yeast Extract | 1-10 | g |
| 09 | KOH | 30-100 | g |
| 10 | Solution of micronutrients*¹ | 1-10 | mL |
| 11 | Fe-EDTA | 0.01-1 | g |
| 12 | Carboxymethyl cellulose (CMC) | 1-10 | g |
| 13 | Saccharose | 10-100 | g |
| 14 | Gelatin | 0.1-1 | g |
| 15 | Mannitol | 1-10 | g |
| 16 | Glycerol | 5-10 | mL |

| | | |
|---|---|---|
| 17 | Water | q.s. |

q.s.: quantum sufficit
*¹see table 4.

TABLE 4
STOCK SOLUTION OF MICRONUTRIENTS
AND QUANTITY NEEDED FOR 1 L OF
FINAL SOLUTION OF MICRONUTRIENTS.

| | | 1 L | | 1 L | |
|---|---|---|---|---|---|
| | Reagents | (stock solution) | | (micronutrients) | |
| 01 | (NH₄)₆Mo₇O₂₄ | 100-200 | g | 84 | mL |
| 02 | MnSO₄ | 100-300 | g | 84 | mL |
| 03 | H₃BO₃ | 50-150 | g | 648 | mL |
| 04 | CuSO₄ | 1-10 | g | 84 | mL |
| 05 | ZnSO₄ | 1-10 | g | 84 | mL |

### EXAMPLES

### Example 1—Grading of Culture

The A. brasilense and P. fluorescens are inoculated separately in flasks containing 100 mL of culture medium NFb (Dobereiner, 1995) and 4.1 as described in Table 1, being incubated in orbital agitator of 80-200 rpm, at 22 to 38° C. for approximately 18 to 96 hours. The following step of grading consists of the inoculation of inox flasks containing 10 L of culture medium as described in Table 2 and Table 1, for the microorganisms A. brasilense and P. fluorescens, respectively, the species are cultivated separately and incubated for approximately 18 to 96 hours, with air flow 0.25 to 1.0 Nm³/h (=0.45-2.5 vvm) and temperature approximately 22-38° C.

Once this time has expired, each culture containing two inox flasks with 10 L of culture medium is inoculated in a tank containing about 180 L of specific culture medium for each microorganism, using the culture medium in Table 2 the specific culture medium for the A. brasilense; and in Table 3 the specific culture medium for P. fluorescens and incubated for approximately 24 to 168 hours, with air flow 1.0 to 10.0 Nm³/h (=0.1-0.83 vvm) and temperature varying from 22 to 38° C.

### Example 2—Mixture of Azospirillum and Pseudomonas in Bioreactor

For the mixture of the microorganisms in a fermenter of 2000 L, preferably the process of sterilization uses a volume of 1600 L of culture medium (Table 3) and is carried out for approximately 60 to 120 minutes, at a temperature of approximately 121° C. to approximately 130° C. Preferably, sterilization is carried out at a pressure of approximately 1.0-2.0 Kgf/cm 2. Preferably, after the sterilization process, a tank of A. brasilense is then inoculated and fermented for about 18 to about 120 hours. The air flow is preferably about 1.0 Nm³/h to about 2.5 Nm³/h (=0.0085-0.021 vvm). The pressure is preferably about 1.0 to about 1.2 kgf/cm 2. The agitation is preferably about 40 hz to about 45 hz. The stabilization process of the inoculant product that enables the mixture of the microorganisms is preferably carried out with a tank containing 200 L of the culture of P. fluorescens is inoculated to the fermenter for approximately about 1 h to 2 h. Preferably, the final product is bottled in sterile bags.

### Example 3—the Mixture of Different Genera of Microorganisms Enables Increased Shelf-Life of the Agricultural Composition

Although the microorganisms generally grow in pH near to neutral, they present different characteristics when cultivated in different sources of carbon (C). When cultivated using malic acid as main source of carbon (Dobereiner, 1995), Azospirillum causes an increase in the pH of the medium that assumes an alkaline content, whereas strains of Pseudomonas, when cultivated with sucrose or glycerol as sources of carbon, they acidify the culture medium. This imbalance in the pH of the culture medium may anticipate the cellular death phase of these microorganisms cultivated in isolation.

Preferably, the formulation of A. brasilense cultivated in isolation uses as main sources of carbon or mannitol, the glycerol and the sucrose, essential sources for the production of exopolysaccharides during the fermentative process. In this embodiment of culture, the average of the pHs of the batches stored is 7.1 (Table 5) and the viability of the cells, after 7 months of storage, drastically falls, increasing from 1.0×10 9 UFC/mL to about 3.3×10 7 UFC/mL, a reduction of 97% in cellular viability. In the same way, when the P. fluorescens is cultivated and bottled individually, after the same seven months of storage, the concentration of viable cells is reduced by 97.7%, increasing from 2.5×10 9 UFC/mL to about 5.6×10⁷ UFC/mL (Table 6), with the initial pH of about 6.92 acidified to 5.22. In contrast, when the product is made up of the consortium between Azospirillum and Pseudomonas, the average of the pH of the batches stored is 6.2 (Table 7). Unexpectedly, when the two microorganisms are mixed in a single product, synergy occurs, enabling the stability of the cells of the two genera employed for prolonged storage periods of up to 18 months as observed in Table 7; initial concentrations of about 1.0×10⁹ UFC/mL undergo a slight reduction to about 7.7×10⁸ UFC/mL after 12 months of storage, a reduction of just 33% in the viability of the cells in a longer storage period. The production of the organic acids by Pseudomonas fluorescens begins after the process of stabilizing the mixture of the microorganisms during the storage period, since Pseudomonas fluorescens consumes the exopolysaccharide produced by Azospirillum brasilense during the fermentative process.

TABLE 5
CONCENTRATION OF CELLS OF AGRICULTURAL
COMPOSITIONS CONTAINING AZOSPIRILLUM
STORED FOR DIFFERENT TIME PERIODS.

| | | | CONCEN- | | | |
|---|---|---|---|---|---|---|
| | INITIAL | | TRATION | | | |
| | CONCEN- | | AFTER | | | |
| | TRATION | initial | STORAGE | pH AFTER | STORAGE | |
| BATCH | (UFC/mL) | pH | (UFC/mL) | STORAGE | TIME | |
| A | 3.10 × 10⁹ | 6.82 | 6.0 × 10⁷ | 7.21 | 7 | months |
| B | 2.39 × 10⁹ | 6.75 | 3.0 × 10⁷ | 7.10 | 7 | months |
| C | 1.30 × 10⁹ | 7.30 | 2.1 × 10⁷ | 7.18 | 8 | months |
| D | 1.52 × 10⁹ | 6.91 | 5.0 × 10⁷ | 7.26 | 9 | months |
| E | 1.65 × 10⁹ | 6.91 | 2.8 × 10⁷ | 7.30 | 10 | months |
| F | 1.82 × 10⁹ | 6.97 | 4.0 × 10⁷ | 7.95 | 10 | months |
| G | 1.31 × 10⁹ | 7.04 | 1.3 × 10⁷ | 6.93 | 10 | months |
| H | 2.00 × 10⁹ | 6.90 | 3.1 × 10⁷ | 7.10 | 10 | months |
| I | 3.60 × 10⁹ | 6.98 | 2.0 × 10⁷ | 6.97 | 11 | months |
| J | 1.51 × 10⁹ | 7.05 | 4.5 × 10⁷ | 6.96 | 12 | months |

TABLE 6
CONCENTRATION OF CELLS OF INOCULANTS CONTAINING
PSEUDOMONAS STORED FOR DIFFERENT TIME PERIODS.

| | INITIAL | | | | |
|---|---|---|---|---|---|
| | CONCEN- | | CONCEN- | | |
| | TRATION | | TRATION | | STORAGE |
| BATCH | (UFC/mL) | pH | (UFC/mL) | pH | TIME |
| 1 | 3.1 × 10⁹ | 6.95 | 5.2 × 10⁷ | 5.22 | 8 months |
| 2 | 2.5 × 10⁹ | 6.97 | 5.0 × 10⁷ | 5.12 | 7 months |
| 3 | 1.9 × 10⁹ | 6.89 | 6.6 × 10⁷ | 5.34 | 7 months |

TABLE 7
CONCENTRATION OF CELLS OF AGRICULTURAL COMPOSITIONS COMPOSED BY AZOSPIRILLUM
AND PSEUDOMONAS IN A SINGLE PACKAGE STORED FOR DIFFERENT TIME PERIODS.

| | INITIAL CONCENTRATION | | | CONCENTRATION | | | |
|---|---|---|---|---|---|---|---|
| | (UFC/mL) | | | (UFC/mL) | | | STORAGE |
| BATCH | Azospirillum | Pseudomonas | pH | Azospirillum | Pseudomonas | pH | TIME |
| 1 | 1.9 × 10 [symbol] | 4.1 × 10 [symbol] | 6.89 | 6.6 × 10 [symbol] | 1.8 × 10 [symbol] | 6.11 | 6 months |
| 2 | 1.4 × 10 [symbol] | 1.3 × 10 [symbol] | 7.04 | 8.5 × 10 [symbol] | 1.5 × 10 [symbol] | 6.47 | 10 months |
| 3 | 2.3 × 10 [symbol] | 1.8 × 10 [symbol] | 7.05 | 6.6 × 10 [symbol] | 3.5 × 10 [symbol] | 6.35 | 10 months |
| 4 | 1.6 × 10 [symbol] | 1.6 × 10 [symbol] | 6.94 | 9.5 × 10 [symbol] | 1.0 × 10 [symbol] | 6.25 | 10 months |
| 5 | 1.4 × 10 [symbol] | 1.8 × 10 [symbol] | 6.95 | 7.4 × 10 [symbol] | 1.0 × 10 [symbol] | 6.10 | 10 months |
| 6 | 2.2 × 10 [symbol] | 5.4 × 10 [symbol] | 6.72 | 1.05 × 10 [symbol] | 2.2 × 10 [symbol] | 6.04 | 10 months |
| 7 | 1.6 × 10 [symbol] | 5.0 × 10 [symbol] | 6.87 | 8.3 × 10 [symbol] | 1.7 × 10 [symbol] | 6.12 | 12 months |
| 8 | 2.3 × 10 [symbol] | 1.1 × 10 [symbol] | 6.95 | 3.0 × 10 [symbol] | 9.0 × 10 [symbol] | 6.22 | 18 months |

[symbol]

indicates data missing or illegible when filed

Example 4—Synergy of the Azospirillum and Pseudomonas enables increased stability and cellular viability for longer storage periods.

The combination of the microorganisms of different genera in a single commercial product with different action mechanisms, such as promoting growth in plants by the biosynthesis of phytohormones or else by solubilization of phosphorus is of fundamental importance for more sustainable agriculture.

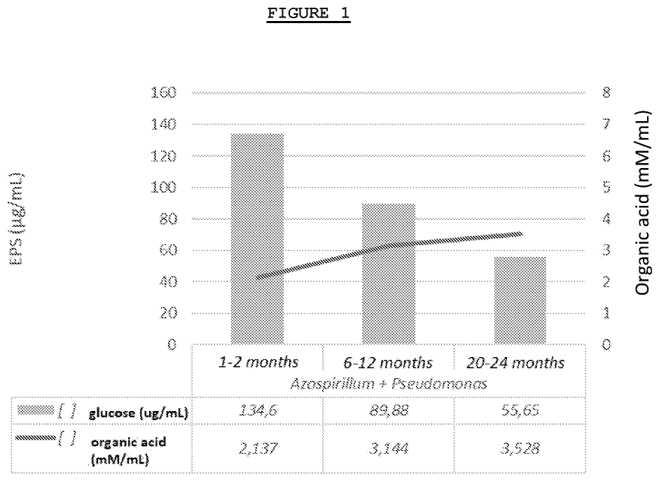

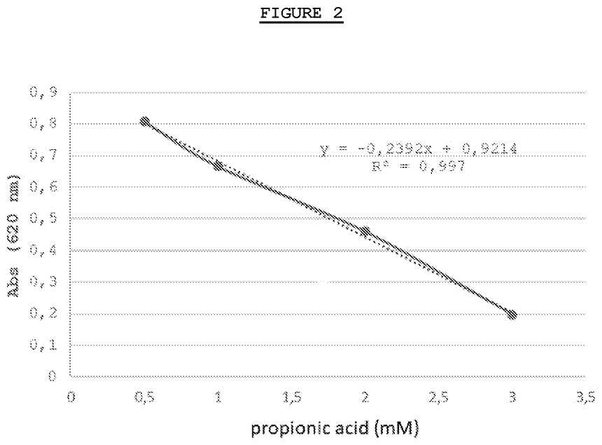

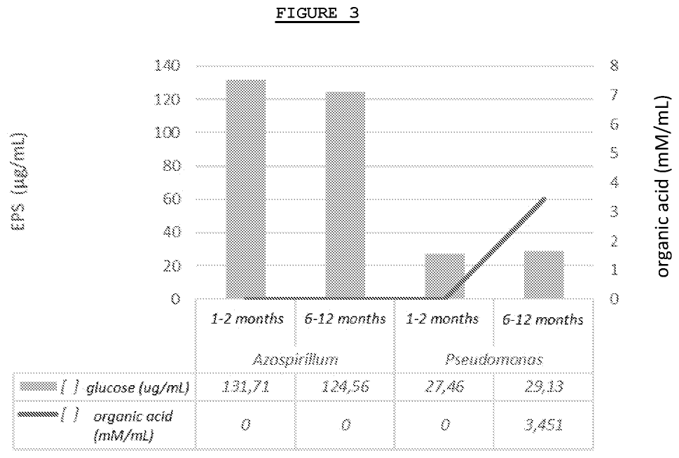

However, the major challenge of this combination in a single product with high stability and cellular viability is associated to growth metabolism, for example, the use of the different sources of carbon, culture time, pressure, temperature, aeration and, even the particular aspects possessed by each genus. For the bacteria of the genus Pseudomonas, as mentioned in example 3, the reduction in the pH during the culture and storage is related to the production of organic acids as demonstrated in FIG. 1.

For quantifying the biosynthesis of organic acids by Pseudomonas, an indirect colorimetric method was developed to evaluate the quantification of these compounds. In this context, a calibration curve was initially made using different gradients of propionic acid (0.5-4 mM) as an indirect method of quantifying the production of organic acids produced by microorganisms (FIG. 2).

Quantifying the production of organic acids was made by spectrophotometer at 620 nm, using the solution of bromothymol blue 0.5% in absolute alcohol as colorimetric method. This compound is an indicator of pH, that is, when added to a more acidic culture medium, the coloring becomes yellow whereas in the more alkaline culture medium the coloring is blue.

To evaluate the samples, the inoculants were centrifuged at 10,000 rpm for 10 minutes, in order to separate the microbial biomass, aliquots of 2 mL of each supernatant were added 20 μL of the bromothymol blue solution and three-fold readings were taken.

The aliquots of the products containing just Pseudomonas stored for 1 to 2 months did not present detectable amounts of organic acids, whereas with longer storage periods (6 to 12 months), the presence of 3.45 mM/mL of organic acids was detected. This high quantity of organic acids caused a strong acidification of the medium, leading to a loss in the cellular viability of 97.7%.

Regarding the biosynthesis of EPS, Pseudomonas fluorescens did not demonstrate capacity to produce it during the course of storage studied.

Contrary to that which occurs with Pseudomonas, for Azospirillum it is possible to note (FIG. 1) the high concentration of EPS independent of the storage period and the absence of production of organic acids. Commonly recognized by the importance in protecting different embodiments of abiotic stress, the EPS produced by Azospirillum were not important for maintaining the cellular viability of this microorganism when bottled individually (Table 5).

Surprisingly, when the two microorganisms were mixed in a consortium agricultural composition, it was possible to establish the increased stability and cellular viability of both the microorganisms, Azospirillum and Pseudomonas (Table 7). The mixture of the two microorganisms enables the prolonged storage of 18 months with an initial count of 1×10⁹ to 3×10⁹ numbers of cells of Azospirillum brasilensis and from 1×10⁷ to 5×10⁷ numbers of cells of Pseudomonas fluorescens, and after 18 months, attains 1×10⁸ to 1×10⁹ number of cells of Azospirilum brasilensis and from 9×10⁷ to 5×10⁸ number of cells of Pseudomonas fluorescens, in a temperature range of 5-40° C.

Unexpectedly, synergism occurs between Azospirillum and Pseudomonas which is explained by; i. High biosynthesis of EPS by Azospirillum; ii. Consumption, by Pseudomonas, of the EPS produced by Azospirillum; biosynthesis of organic acids by Pseudomonas; consumption, by Azospirillum, of the organic acids synthesized by Pseudomonas (FIGS. 1 and 3). This biosynthesis and consumption dynamics is shown in FIGS. 1 and 2, where the EPS produced by Azospirillum is gradually consumed by Pseudomonas during the course of the storage time. It is noted in this case that the EPS are synthesized during fermentation. The organic acids synthesized during the course of storage by Pseudomonas tend to assume stable concentrations, since, in parallel to biosynthesis, it is consumed by Azospirillum, which is reaffirmed by the maintenance of the pH of the consortium agricultural composition over time.

### REFERENCES

Altenbach SB New insights into the effects of high temperature, drought and post-anthesis fertilizer on wheat grain development. J Cereal Sci v. 56, n. 1, p. 39-50.
Ajar Nath Yadav, Priyanka Verma, Bhanumati Singh, Vinay Singh Chauahan, Archna Suman and Anil Kumar Saxena. Plant Growth Promoting Bacteria: Biodiversity and Multifunctional Attributes for Sustainable Agriculture. Adv Biotech & Micro. v. 5, n. 5 2012.
Baby Shaharoona, Muhammad Arshad, Zahir A. Zahir, Azeem Khalid, Performance of Pseudomonas spp. containing ACC-deaminase for improving growth and yield of maize (Zea mays L.) in the presence of nitrogenous fertilizer, Soil Biology and Biochemistry, v. 38, n. 9, p. 2971-2975, 2006.
Brion K. Duffy, Genevieve Defago. Environmental Factors Modulating Antibiotic and Siderophore Biosynthesis by Pseudomonas fluorescens Biocontrol Strains. Applied and Environmental Microbiology, p. 2429-2438, 1999.
Bric, J. M.; Bostock, R. M.; Silverstone, S. E. Rapid in situ assay for indoleacetic acid production by bacteria immobilized on a nitrocellulose membrane. Appl Environ Microbiol v. 57, n. 2, p. 535-538, 1991.
Brown, M. E.; Burlingham, S. K. Production of plant growth substances by Azotobacter chroococcum. J Gen Microbiol v. 53, n. 1, p. 135-144, 1968.
Boddey, R.; De Oliveira, O.; Urquiaga, S.; Reis, V.; De Olivares, F. et al. Biological nitrogen fixation associated with sugar cane and rice: contributions and prospects for improvement. Plant Soil v. v. 174, n. 1-2, p. 195-209, 1995.
Bakker, A. W.; Schippers, B. Microbial cyanide production in the rhizosphere in relation to potato yield reduction and Pseudomonas SPP-mediated plant growth-stimulation. Soil Biol Biochem v. 19, n. 4, p. 451-457, 1987.
Cappucino, J. C.; Sherman, N. Nitrogen Cycle. In: Microbiology: A Laboratory Manual. (4th edn), Benjamin/Cumming Pub Co, New York, USA, p. 311-312, 1992.
Cassán, F. et al. Protocolo para el control de calidad de inoculantes que contienen Azospirillum sp. Documento de Procedimientos de la REDCAI (Red de Control de Calidad de inoculantes, n. 2, la ed. Buenos Aires: Associacion Argentina de Microbiologia, 2010. 13 pp. CD-ROM.
Dorien M. Kool, Jan Dolfing, Nicole Wrage, Jan Willem Van Groenigen, Nitrifier denitrification as a distinct and significant source of nitrous oxide from soil, Soil Biology and Biochemistry, v. 43, n. 1, p. 174-178, 2011.
Duca, D. R.; Rose, D. R.; Glick, B. R. Indole acetic acid overproduction transformants of the rhizobacterium Pseudomonas sp. UW4. Antonie van Leeuwenhoek v. 111, p. 1645-1660, 2018.
Fasim, F.; Ahmed, N; Parsons, R.; Gadd, G. M. Solubilization of zinc salts by a bacterium isolated from the air environment of a tannery. FEMS Microbiol Lett v. 213, n. 1, p. 1-6, 2002.
Hill, G.; Mitkowski, N.; Aldrich-Wolfe, L.; Emele, L.; Jurkonie, D.; Ficke, A.; Maldonado-Ramirez, S.; Lynch, S.; Nelson, E. Methods for assessing the composition and diversity of soil microbial communities. Appl Soil Ecol v. 15, n. 1, p. 25-36, 2000.
Hu X, Chen J, Guo J. Two Phosphate- and Potassium-solubilizing Bacteria Isolated from Tianmu Mountain, Zhejiang, China. World J Microbiol Biotechnol v. 22, n. 9, p. 983-990, 2006.
Jacobson, C. B.; Pasternak, J.; Glick, B. R. Partial purification and characterization of 1-aminocyclopropane-1-carboxylate deaminase from the plant growth promoting rhizobacterium Pseudomonas putida GR12-2. Can J Microbiol v. n. 12, 1019-1025, 1994.
Molina, R., Rivera, D., Mora, V. et al. Regulation of IAA Biosynthesis in Azospirillum brasilense Under Environmental Stress Conditions. Curr Microbiol. v. 75, p. 1408-1418, 2018.
King, E. O.; Ward, M. K.; Raney, D. E. Two simple media for the demonstration of pyocyanin and fluorescin. The Journal of Laboratory and Clinical Medicine, v. 44, p. 301-307, 1954.
Kopycinska, M.; Lipa, P.; Cie61a, J.; Koziel, M.; Janczarek, M. Extracellular polysaccharide protects Rhizobium leguminosarum cells against zinc stress in vitro and during symbiosis with clover. Environmental and microbiology reports. 2018.
Kumar, V.; Yadav, A. N.; Verema, P.; Sangwan, P.; Abhishake, S.; et al. 13-Propeller phytases: Diversity, catalytic attributes, current developments and potential biotechnological applications. Int J Biol Macromolec v. 98, p. 595-609, 2017.
Kloepper, J.; Schroth, M. Plant growth-promoting rhizobacteria on radishes. In: Proceedings of the 4th international conference on plant pathogenic bacteria, p 879-882, 1978.
Pikovskaya, R. Mobilization of phosphorus in soil in connection with vital activity of some microbial species. Mikrobiologiya 17: 362-370, 1948.
Suman, A.; Verma, P.; Yadav, N. A.; Saxena, A. K. Bioprospecting for extracellular hydrolytic enzymes from culturable thermotolerant bacteria isolated from Manikaran thermal springs. Res J Biotechnol v. 10, n. 33-42, 2015.
Suman, A; Verma, P.; Yadav, A. N.; Srinivasamurthy, R.; Singh, A.; Prasanna, R. Development of hydrogel based bio-inoculant formulations and their impact on plant biometric parameters of wheat (Triticum aestivum L.). Int J Curr Microbiol Appl Sci. v. 5, n. 3, p. 890-901, 2016.
Schwyn, B.; Neilands, J. Universal chemical assay for the detection and determination of siderophores. Anal Biochem v. 160, n. 1, p. 47-56, 1987.
Verma, P.; Yadav, A. N.; Shukla, L.; Saxena, A. K.; Suman, A. Hydrolytic enzymes production by thermotolerant Bacillus altitudinis IARI-MB-9 and Gulbenkiania mobilis IARI-MB-18 isolated from Manikaran hot springs. Int J Adv Res. V. 3, n. 9, p. 1241-1250, 2015.
Verma, P.; Yadav, A. N.; Khannam, K. S.; Kumar, S.; Saxena, A. K.; Suman, A. Molecular diversity and multifarious plant growth promoting attributes of Bacilli associated with wheat (Triticum aestivum L.) rhizosphere from six diverse agro-ecological zones of India. J Basic Microbiol v. 56, n. 1, p. 44-58, 2006.
Yadav A N, Sharma D, Gulati S, Singh S, Kaushik R, et al. Haloarchaea endowed with phosphorus solubilization attribute implicated in phosphorus cycle. Sci Rep. 2015.
Yadav, A. N.; Verma, P.; Kumar, V.; Sachan, S. G.; Saxena, A. K. Extreme Cold Environments: A Suitable Niche for Selection of Novel Psychrotrophic Microbes for Biotechnological Applications. Adv Biotechnol Microbiol v. 2, n. 2, p. 1-4, 2017.

1.-34. (canceled)

35. An agricultural composition composed of a mixture of Azospirillum and Pseudomonas microorganisms as an inoculant product promoting plant growth, comprising: Azospirillum brasilense in a concentration of 1.0×10⁸ UFC/mL to 3.0×10⁹ UFC/mL and Pseudomonas fluorescens in a concentration of 1.0×10⁷ to 5.0×10⁸ UFC/mL of inoculant;
wherein the microorganisms propagate at low temperatures, below 20° C., in soils with low pH, below 6.0;
the agricultural composition contains water, sucrose, carboxymethyl cellulose, gelatin and mannitol as carrier; and
the mixture promotes prolonged storage time.

36. The agricultural composition of claim 35, wherein the prolonged storage time is 18 months, with an initial count of 1×10⁹ to 3×10⁹ numbers of cells of Azospirillum brasilense and 1×10⁷ to 5×10⁷ number of cells of Pseudomonas fluorescens, and after 18 months, reaches 1×10⁸ to 1×10⁹ number of cells of Azospirillum brasilense and 9×10⁷ to 5×10⁸ number of cells of Pseudomonas fluorescens, in a temperature range of 5-40° C.

37. Industrial process of the mixture of Azospirillum and Pseudomonas as inoculant product promoting plant growth, comprising the steps of:
(a) formulation of a commercial biotechnological inoculant product composed of two genera of microorganisms in a technical solution that enables the mixture of the parts with high stability, presented in a single package; and
(b) stabilization of a biotechnological inoculant product composed by the mixture of Azospirillum and Pseudomonas in a technical solution that enables the mixture of the parts, presented in a single package.

38. The industrial process of the mixture of Azospirillum and Pseudomonas of claim 37, wherein the stabilization process of the product of step (b) is carried out for approximately 1 to 2 hours.

39. The industrial process of the mixture of Azospirillum and Pseudomonas of claim 37, wherein the two genera are selected from the group consisting of Azospirillum brasilense and Pseudomonas fluorescens.

40. The industrial process of the mixture of Azospirillum and Pseudomonas of claim 37, wherein the microorganisms are mixed in fermenters, and the step of mixture of Azospirillum and Pseudomonas is carried out during about 1 to about 2 hours.

41. The industrial process of the mixture of Azospirillum and Pseudomonas of claim 37, wherein the fermentation of the culture is by batch;
the fermentation process is carried out at a temperature of approximately 22° C. to 38° C.;
the fermentation process is carried out at an air flow of approximately 1.0 Nm³/h to approximately 2.5 Nm³/h or 0.0085-0.021 vvm.

42. The industrial process of the mixture of Azospirillum and Pseudomonas of claim 37, further comprising the sequential expansion of the culture of different genera of bacteria for inoculating the fermentation culture, and the sequential expansion is made in volumes of about 100 mL, to about 10 L, about 180 L to about 2000 L.

43. The industrial process of the mixture of Azospirillum and Pseudomonas of claim 37, wherein the bacteria genera are inoculated separately.

44. The industrial process of the mixture of Azospirillum and Pseudomonas of claim 37, wherein the genera of bacteria are expanded by incubation in an orbital agitator to about 80 rpm to about 200 rpm;
the genera of bacteria are expanded by incubation for about 18 hours to about 96 hours;
the genera of bacteria are expanded in inox flasks containing about 10 L of culture medium;
the genera of bacteria are incubated for approximately about 18 to about 96 hours;
the genera of bacteria are incubated with air flow of about 0.25 Nm³/h to about 1.0 Nm³/h or 0.45-2.5 vvm for 10 L of culture medium, for approximately 18-96 hours and temperature approximately 22-38° C.

45. The industrial process of the mixture of Azospirillum and Pseudomonas of claim 44, wherein the genera of bacteria after the culture segregated in two inox flasks of about 10 L, said two flasks are inoculated in tanks containing about 180 L of culture medium;
the strains are incubated for about 24 to about 168 hours;
the genera of bacteria are incubated with air flow of about 1.0-10.0 Nm³/h or about 0.1-0.83 vvm; and
the incubation temperature is from about 22° C. to about 38° C.

46. The industrial process of the mixture of Azospirillum and Pseudomonas of claim 37, wherein the fermentation step is carried out with pressure of about 1.0 to about 1.2 kgf/cm³;
the fermentation step is carried out with agitation of about 40 hz to about 45 hz;
the fermentation step is carried out at a temperature of about 22° C. to about 38° C.;
the fermentation step is carried out with air flow of about 1.0 Nm³/h to about 2.5 Nm³/h or about 0.0085-0.021 vvm.

47. The industrial process of the mixture of Azospirillum and Pseudomonas of claim 37, wherein production occurs of exopolysaccharides (EPS) in Azospirillum, and of the biosynthesis of organic acids by Pseudomonas.

48. The industrial process of the mixture of Azospirillum and Pseudomonas of claim 37, wherein a mixture of microorganisms is applied in amounts of 150 mL/ha via seed, 300 to 1000 mL/ha via plantation furrow and 500 to 1000 mL/ha via leaf spraying.

49. Use of agricultural composition composed by a mixture of Azospirillum and Pseudomonas as inoculant product promoting plant growth, as defined in claim 35, wherein it is used for application in various agricultural crops, for application via seed, furrow and post-emergence spraying, and for promoting plant growth and solubilizing phosphorus.
